(19) United States
(12) Patent Application Publication
(10) Pub. No.: US 2024/0041391 A1
(43) Pub. Date: Feb. 8, 2024

(54) Method and Apparatus for Orthopedic Procedure State Indication

(71) Applicants: Kenneth Anderson Stewart, Madison, WI (US); Robert Neil Shaddock, San Francisco, CA (US); Anya Chambers, San Francisco, CA (US); Nicholas Mast, San Rafael, CA (US)

(72) Inventors: Kenneth Anderson Stewart, Madison, WI (US); Robert Neil Shaddock, San Francisco, CA (US); Anya Chambers, San Francisco, CA (US); Nicholas Mast, San Rafael, CA (US)

(21) Appl. No.: 18/364,284

(22) Filed: Aug. 2, 2023

Related U.S. Application Data
(60) Provisional application No. 63/394,762, filed on Aug. 3, 2022.

(51) Int. Cl. A61B 5/00; A61B 17/16

(52) U.S. Cl. CPC A61B 5/4504; A61B 17/1626; A61B 2017/00022

(57) ABSTRACT

A method and apparatus are described for processing sensor and data during an orthopedic procedure to analyze and report on the state of the bone structure surrounding a surgical site. This may include, for example, the state of the femoral canal during the broaching phase of a total hip arthroplasty procedure. The method and apparatus allow a surgeon to determine, amongst other things, the optimality of fit of the broaching instrument, and subsequently of the implanted prosthesis, within the femoral canal, with consequential enhancement to patient outcome and reduction in economic cost.

### CROSS-REFERENCE TO RELATED APPLICATIONS

This application claims priority to U.S. Patent Application No. 63/394,762 filed Aug. 3, 2022, the entire contents of which are incorporated here by reference.

### BACKGROUND OF THE INVENTION

As global populations age, the number of hip replacements, or total hip arthroplasty (THA) procedures executed annually are steadily increasing. There are a variety of underlying conditions that motivate a THA, including osteoarthritis, rheumatoid arthritis, and osteonecrosis. Whatever the reason, THA's alleviate the pain and progressive debilitation associated with these conditions by replacing the defective hip joint with an artificial hip joint or prosthesis. The increasing annual frequency of THA's also means that the total economic cost of such procedures is steadily rising.

As the number of THA procedures undertaken annually increases, so does the importance of executing such procedures safely and efficiently. Safety implies excellent patient outcomes, minimization of any complications that might occur during surgery, and good restoration of hip joint function with minimum recovery time. At the same time, to control costs, it is important to optimize the efficient use of operating room (OR) resources, including staff and material, and to avoid wherever possible further intervention to correct problems incurred during an initial THA. It is important therefore to minimize the likelihood that any aspect of the THA procedure is sub-optimally or incorrectly executed. Such events can have a negative impact on patient outcomes, as well as incurring additional economic costs in terms of both further restorative surgical intervention and prolonged recovery periods.

As a result of these factors, especially in the case of surgeons who are inexperienced with THA procedures, a method or apparatus which can reduce the likelihood of sub-optimal THA procedure execution would benefit not only patient outcomes but also reduce the associated economic cost, for the patient, insurer and even the institution hosting the THA procedure.

While most THA procedures are executed successfully, there can be negative outcomes due to infection, prosthesis mis-sizing and loosening, and so on. The basis for several important sources of failure is rooted within the procedure itself. Complications may occur, for example, during the phase when the surgeon is preparing the femur to receive the implanted prosthesis. During this phase, the surgeon will prepare the femoral canal to permit insertion of the prosthesis by either compacting or extracting via rasping the material lying within the canal. This preparation is usually executed by using a mechanical reaming tool often referred to as a broach. The broach tool can be inserted into the femoral canal by manual hammering with a mallet or similar tool or by using an automated hammering device such as an electro-mechanical impactor.

It is during this phase of broaching that the femoral wall may become over-stressed or under-stressed by the reaming or broaching process, leading to a variety of problems. These can include mechanical loosening of the prosthesis in the case where the broaching process is insufficient, or where the prosthesis dimension is poorly matched to the femoral cavity created by broaching. Alternatively, over-broaching can lead to fracture of the femur during surgery (an intraoperative fracture) or during patient recovery (post-operatively). Both under- and over-broaching can therefore have grave consequences for the patient, requiring further surgical intervention, extending patient risk and recovery time, and incurring unintended economic cost.

Accordingly, any method or apparatus which can assist the surgeon executing a THA to apply an optimal degree—i.e., neither too much nor too little—of broaching would be highly beneficial to patient outcomes and cost reduction. Such a method or apparatus must be able to operate contemporaneously or in “real-time” during the procedure as the surgeon works. It must also be able to provide critical feedback on broaching degree to assist the surgeon during the procedure without causing distraction.

It is known that the femoral broaching process can be accompanied by a variety of visual, haptic, tactile (including vibrational) and acoustic phenomena. When interpreted by a skilled surgeon, these can indicate when the correct or optimal degree of broaching has occurred. For example, as the broaching process approaches the optimal point, the surgeon may perceive—either aurally, tactilely, or haptically—that each impact with the mallet or automated impactor results in a “ping” or other characteristic acoustic, visual or vibrational phenomena. That is, a characteristic acoustic, visual, or tactile “signature” can occur which indicates the degree of broaching, and which can therefore be used to determine an optimal degree of broaching. A skilled surgeon sometimes uses such impact signatures to guide his or her broach impact process, including decisions such as when to terminate broaching, when to change broach dimensions, extract the broach and so on. Often, such skill is developed over hundreds or even thousands of surgical procedures and can take many years to accrue.

Accordingly, a method or apparatus which enhances the skill and judgement of surgeons to interpret such visual, acoustic, or tactile impact signatures or signals and provide an indication of broaching optimality would be a valuable surgical aid, especially to less experienced surgeons and surgical teams, surgeons in training, or to surgeons who only infrequently execute a THA or similar procedure.

Any such method or apparatus should have low cost and, given the surgical setting, should be also easy to sterilize or avoid risk of infection entirely by being displaced from the surgical site and located outside of the sterile field surrounding that site. A natural approach here is to process acoustic emissions from the broaching process, that is to use one or more microphones as sensors.

This was the approach taken, for example, in U.S. Patent Publication No. 2017/0112634 A1 entitled “Objective, Real-Time Acoustic Measurement and Feedback for Proper Fit and Fill of Hip Implants” by Gunn et al., the entirety of which is incorporated herein by reference. The publication describes a method for using acoustic data to determine proper fit of a hip implant. After transferring data to a control unit, the publication describes first segmenting the acoustic data into sequences of impacts (US 2017/0112634, FIG. 3, step 230) by detecting a region of four seconds in which the audio exceeds 50% of the amplitude limit to which the microphone is tuned. Individual impacts within the sequence are then identified by forming the signal envelope, decimating by a factor of 100×, and then forming a signal envelope estimate. An individual impact is segmented and declared if the resulting processed signal envelope falls below a specified threshold. The publication further describes taking a single discrete Fourier transform (DFT) of each so-identified impulse and then processing features of the resulting frequency-domain representation of the impulse using a support vector machine (SVM) or kernel machine.

The publication further describes that after each impact is detected, the audio is subsequently transformed (US 2017/0112634, FIG. 3, step 240) into length-3 frequency-domain features. These frequency domain features are defined as signal power in the 1-2 kHz, 2-4 kHz and 5-7 kHz frequency bands, respectively. Possible additional features include power in lower bands, decay rates of both the signal and of specific harmonic regions, zero-crossing rates and cepstral analysis. The formation of Mel frequency cepstral coefficients (MFCC) is also described, but there appears to be no specific information on what should be done with the resulting MFCCs.

US2017/0112634 further describes an optional process of classification training (US 2017/0112634, FIG. 3, step 250) where the previously defined 3-dimensional feature vector is used as the basis for supervised learning. An optimal separating plane is created that lies between the 3-dimensional feature vector points corresponding to impacts observed before a final fit, and those observed after a final fit. The separating plane is created using a soft-threshold support vector machine (SVM). Thereafter, impacts may be binary classified either as a ‘good fit’ or ‘poor fit’. The publication further states that this binary “good” or “bad” fit classification may further be assigned a confidence measure according to the distance from the separating plane of the endpoints of the 3-dimensional vectors associated with each new impact.

The approach described in US 2017/0112634 suffers, however, from several important drawbacks. First, in a busy operating theatre, there are many events which can lead to acoustic or audio emissions in addition to broach impacts. These include preparatory orthopedic operations such as staff movement or conversation, emissions from patient vital sign monitors, chiseling or sawing operations, tool preparation, tool or tissue disposal, and so on. Many of these events lead to elevated audio levels or possess envelopes which appear in the time domain to be very similar to those of broach impacts. Accordingly, many ordinary but non-impact events in the operating room can be incorrectly identified as impacts when using envelope detection. A method that can discriminate between different types of audio or acoustic event in the operating room is desired to ensure only acoustic emissions related to impacts are included for subsequent broach or prosthesis fit estimation.

Further, even the acoustic emissions attributable only to impacts may possess important variations. For example, the surgeon may use one or more different types of manual mallet or automatic impact devices to produce impacts, including mallets or devices from different vendors. Indeed, even within the same procedure, the surgeon may switch between manual and automatic impactors. While these can appear similar in the time domain and from the perspective of signal envelope, it is essential to identify and discriminate the type of tool being used to execute each impact and impact sequence before passing the corresponding data to fit estimation processing. It may be desirable to extract the maximum amount of information from each impact, including information that does not correspond directly to a specific predefined frequency band. Rather, determination of the nature of an impact may depend on information embedded within the entire time-frequency representation of an individual impact rather than only within specific frequency bands or sub-bands.

A further critical consideration is the indication of femoral broach or prosthesis fit that is offered to the surgeon. US 2017/0112634 provides a binary indication of fit—that is, the classified fit indication is of either a ‘poor’ fit or ‘good’ fit, potentially augmented with a confidence measure. This is consistent with, and a limitation of, using a scalar vector machine or kernel machine whose decision regions are designed to deliver only the binary “before” and “after” fit decision. In practice, in order to make the best clinical determination for patients who possess different bone structures including so-called Dorr classification of the femoral bone, age, gender, body mass index, etc., it may be beneficial for surgeons to have a range of fit indications. That is, an indication of degree of fit that spans a defined scale. For example, a 5-point scale, indicating from “very loose fit” to “very tight fit” may be useful. This may be accomplished using specific methods of training — in other words, experienced surgical feedback regarding the degree of fit may be needed to label an impact or a sequence of impacts in a way that can be trained in a supervised learning setting. This exceeds the capability of a binary classifier.

Yet another important consideration is the information contained within the time-sequence of impacts about the evolution of the state of the physical system formed by the combination of the broach and femur. As the broach progresses into the femoral canal, it may encounter regions of temporarily tight fit before dislodging a bony obstruction and then moving further into the canal before re-encountering more bone. This translational motion of the broach can be accompanied by one or more transitory periods of looser fit followed by one or more periods of tighter fit. Consequently, as the surgeon works the broach into the femoral canal, the fit may progress generally from a loose fit to a tight fit, but there may be a wide variation in the tightness measure as the broach progresses and hence also with the acoustic response associated with each individual impact. As a result, it is important that all the information in the preceding or nearby-in-time sequence of impacts comprising the broaching process should be preserved and included as the determination of the tightness of each individual impact is attempted. Further, capturing the information contained within the sequence should not be done with simple averaging methods. Rather, a method that captures the information from each impact, and adds and retains that information within an evolving state representation of the entire impact process may be needed to form accurate estimates of the state of the broaching process and the degree of fit over time.

Note that, in what follows, while the invention is described in terms of a THA procedure, it will be obvious that other procedures where mechanical manipulation of orthopedic or boney structures, or other mechanical or biomechanical systems that result in characteristic acoustical, vibrational, or tactile signatures are also within the scope of the invention.

### SUMMARY OF THE INVENTION

A method and apparatus are described for processing sensor and data during an

orthopedic procedure to analyze and report on the state of the bone structure surrounding a surgical site. This may include, for example, the state of the femoral canal during the broaching phase of a total hip arthroplasty procedure. The method and apparatus allow a surgeon to determine, amongst other things, the optimality of fit of the broaching instrument, and subsequently of the implanted prosthesis, within the femoral canal, with consequential enhancement to patient outcome and reduction in economic cost.

In one embodiment, the method includes one or more of the steps of receiving sensor data, pre-processing the sensor data within time intervals, classifying probabilistically time intervals into defined events, identifying sub-sequences of time intervals comprising events, identifying sequences of events and then identifying metrics from event sequences. In one embodiment, the metric is used to indicate the state of a broaching process during an orthopedic surgical procedure.

### BRIEF DESCRIPTION OF DRAWINGS

Illustrative embodiments of the present disclosure will be described with reference to the accompanying drawings, of which:

FIG. 1 shows the processing architecture, according to one or more embodiments of the present disclosure.

FIG. 2 shows the processing steps, according to one or more embodiments of the present disclosure.

FIG. 3 shows a sequence of acoustic events and associated processing intervals, according to one or more embodiments of the present disclosure.

FIG. 4 shows processing stages, according to one or more embodiments of the present disclosure.

FIG. 5 shows a time frequency representation in isometric format, according to one or more embodiments of the present disclosure.

FIG. 6 shows a time frequency representation as a 2-dimensional image, according to one or more embodiments of the present disclosure.

FIG. 7 shows a sequence of impacts and estimator probabilities, according to one or more embodiments of the present disclosure.

FIG. 8 shows a sequence of detected events, according to one or more embodiments of the present disclosure.

FIG. 9 shows a sequence labelled events, according to one or more embodiments of the present disclosure.

FIG. 10 shows multiple sequences of labelled events, according to one or more embodiments of the present disclosure.

FIG. 11 shows discrete Fourier transformation (DFT) processing, according to one or more embodiments of the present disclosure.

FIGS. 12A and 12B show time-frequency block constructions, according to one or more embodiments of the present disclosure.

FIG. 13 shows multi-source time-frequency block construction, according to one or more embodiments of the present disclosure.

FIG. 14 shows variable-window DFT processing, according to one or more embodiments of the present disclosure.

FIG. 15 shows TIPP interval construction, according to one or more embodiments of the present disclosure.

FIG. 16 shows equalization of the acoustic observation, according to one or more embodiments of the present disclosure.

FIG. 17 shows processing to estimate event probabilities, according to one or more embodiments of the present disclosure.

FIG. 18 shows multi-source event probability estimation, according to one or more embodiments of the present disclosure.

FIG. 19 shows an electro-mechanical impactor, according to one or more embodiments of the present disclosure.

FIG. 20 shows construction of a training event reference location, according to one or more embodiments of the present disclosure.

FIG. 21 shows construction of a reference time interval, according to one or more embodiments of the present disclosure.

FIG. 22 shows event sub-sequence construction, according to one or more embodiments of the present disclosure.

FIG. 23 shows estimation of an event time location, according to one or more embodiments of the present disclosure.

FIG. 24 shows subsequences of event types, according to one or more embodiments of the present disclosure.

FIG. 25 shows separation of event subsequences, according to one or more embodiments of the present disclosure.

FIG. 26 shows association of feature vectors with events, according to one or more embodiments of the present disclosure.

FIG. 27 shows autoencoder processing of time-frequency blocks, according to one or more embodiments of the present disclosure.

FIG. 28 shows gated recurrent unit (GRU) processing, according to one or more embodiments of the present disclosure.

FIG. 29 shows a form of GRU internal architecture, according to one or more embodiments of the present disclosure.

FIG. 30 shows a metric representation to a user interface, according to one or more embodiments of the present disclosure.

FIG. 31 shows an event class hierarchy, according to one or more embodiments of the present disclosure.

FIG. 32 shows impactor event classes, according to one or more embodiments of the present disclosure.

### DETAILED DESCRIPTION OF THE INVENTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

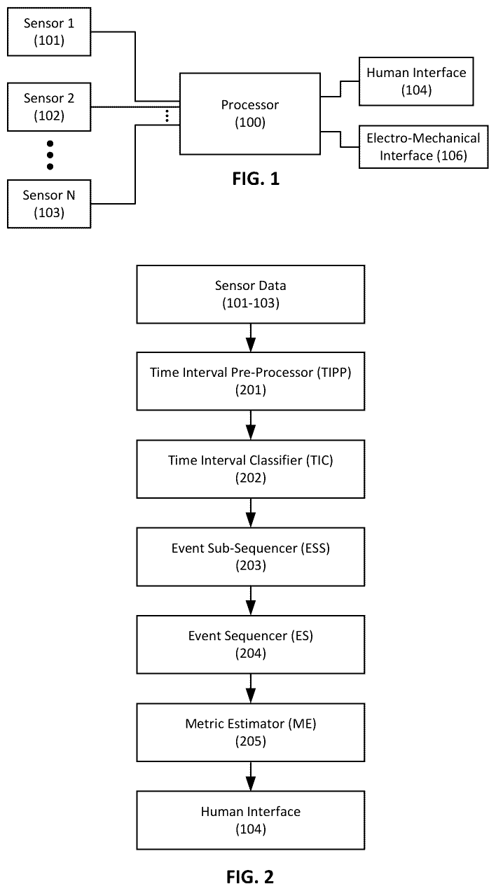

FIG. 1 shows the apparatus of a processing system designed to execute the method, according to one or more embodiments of the present disclosure.

In an embodiment, the system comprises first a series of sensors (101-103) which provide sensed data to a processor (100). These sensors may include, for example, one or more microphones or similar acoustic sensor devices able to detect the acoustic emissions of the broach impact process. The sensors may also include one or more vibration stress or strain sensors mechanically or acoustically coupled to the surgical site, impactor, mallet, broaching tool, implant or prosthesis or other coupled material or structure. In an embodiment, the sensor may be mounted directly onto, or integrated within the coupled material or structure. For example, the sensor may comprise at least a transducer bonded to the surface of a broach tool, prosthesis or other surgical entity using one of many different bonding techniques including adhesives, resins, micro-welding, mechanical fasteners and so on. Further, the transducer may, for example, convert mechanical stress or strain, or acoustic pressure, into a coupled electrical or related signal or property such as a varying resistance, voltage or current.

This electrical signal or property may then be coupled via a wired, wireless, or optical communication transmitter to enable the transfer of a representation of the electrical signal to a receiver and hence to the processor (100). Here, the method of wired, wireless, or optical communication may include a radio frequency (RF), intermediate frequency (IF) or inductively coupled method such as radio frequency identification (RFID) technology. The coupling to the wireless or electromagnetic communication transmitter may be a direct analog coupling (e.g., direct amplitude or frequency modulation responsive to the electrical signal) or may be a digital coupling where the electrical signal is first sampled by an analog-digital converter and then communicated via a digital modulation technique such as frequency shift keying (FSK), minimum shift keying (MSK), quadrature amplitude modulation (QAM) (including binary phase shift keying (BPSK) or quadrature phase shift keying (QPSK)) or any other digital communication technique.

Note that the transducer may be used for other purposes in addition to being a sensor (101-103). For example, the transducer may also be used to determine the post-operative status of a patient.

The processor (100) may be a central processing unit (CPU), digital signal processor (DSP), graphics processing unit (GPU), field programmable gate array (FPGA) or any combination of such devices, including accelerated devices, local or cloud-based virtual processors and so on. The processor typically includes random-access memory (RAM) and/or read-only memory (ROM).

The processor (100) processes the data made available by the sensors (101-103) to generate a representation of the broaching state. In an embodiment, this is done in real-time during the procedure or is generated after a procedure based on recorded data. A potential application of the latter case is to operate in non-real time, potentially using more complex computational or algorithmic operations or using data combined from multiple patients to predict long-term patient outcomes. For convenience, and without loss of generality, we focus here on the case of real-time operation.

During surgery, the surgeon continually monitors the state of the surgical site and adapts his or her strategy to achieve the desired surgical outcome. For orthopedic surgeons, the state of the bone or skeletal structure, as modified by the surgical procedure is generally highly significant. For surgeons executing a THA procedure, the state of the femur during surgery is an example of such a surgical site state. A further example of surgical site state is the state of the femur and the femoral canal during the broaching process. This state of the surgical site is referred to here as the broaching state. The broaching state is a representation of the current state of the femoral broaching process. Here, a term for the broaching state is the broaching state metric (BSM), although other terms may be used. BSM refers to the biomechanical state of the broaching process and may include measures of the deformation of the femoral canal, degree of insertion of the broach, tightness of fit of the broach, stress, or strain level of the femoral wall and so on.

The broaching state metric also permits more narrow or specific definitions or substates of the BSM. The processor (100) may be configured to provide a measure of one or more of these sub-states. For example, the surgeon may be most interested in the tightness of fit of the broach into the femoral canal and may request the processor (100) to provide a form of BSM that is specifically tuned or trained to estimate the tightness of fit of the broach with the canal. In this case, the BSM may be referred to as a tightness index (TI) or Mast tightness index (MTI). For convenience we refer here to the generic BSM designation where it is understood this may also mean any substate of the BSM including MTI.

The BSM is rendered by the human interface (104) in a form interpretable by the surgeon to indicate one or more aspects of the broach state that are of interest to him or her. For example, as stated previously, the surgeon may be interested in the degree of biomechanical tightness of fit of the broach into the femoral canal. In an embodiment, the human interface (104) displays a meter, or array of light emitting diodes arranged in progression to emulate a metering process, or a virtual representation of a meter (e.g., as a virtual object on a computer screen) or other visual representation showing a degree or tightness of fit. An embodiment of such a visual representation appears in FIG. 30, although it will be obvious that other such representations can be readily constructed.

In this instance, the meter may be operable over a pre-calibrated scale of tightness of fit that is known to the surgeon. For example, the BSM might be defined as a tightness index (TI) ranging from zero (0) meaning a very loose fit, to five (5) meaning a very tight fit in the assessment of an experienced surgeon. Additional indices which are not part of the nominal tightness scale—such as the ‘6’ state shown in FIG. 30—can be displayed for the purpose of indicating an ‘under’, ‘over’, or out-of-bounds state. For the purpose of illustration, and without loss of generality, in what follows we frequently focus on tightness index (TI) or equivalently Mast tightness index (MTI) as the exemplary BSM of interest.

Alternatively, a visual human interface (104) may be substituted by, or operated in parallel with, other human interfaces. For example, the human interface (104) might include a tactile or haptic interface, which may issue a vibration towards the surgeon or tighten a wristband worn by the surgeon in response to the BSM. The visual interface can also be displayed on the surface of, or integrated into, the electro-mechanical impactor used to execute broaching.

In another embodiment, the human interface (104) includes an audio interface, where an audio tone is perceptible by the surgeon and where the tone's amplitude, frequency, chirp characteristics or other attribute is responsive to the BSM. Where the BSM or other metric is not needed during surgery—for example, the sensor data acquired during surgery is processed following completion of the procedure—the human interface (104) may be omitted.

Further modes of control of the impactor are also possible, including automatic control modes. In an embodiment, using an electro-mechanical or pneumatic impactor, the impactor may receive from the processor (100) via a wired, wireless, or optical interface (106) a direct or derived representation of the BSM or a control signal responsive to the BSM. This is done using a Wi-Fi, Bluetooth, or other wireless interface, via an optical coupling, or wired cable, or other coupling method. The electro-mechanical impactor then automatically modifies its impacting behavior in response to the received BSM or derived representation or control signal. Here, dynamic behavior includes physical characteristics such as level of impactor recoil, velocity, acceleration, deceleration etc.

In an embodiment, as stated, the impactor may be an electro-mechanical impactor, a pneumatic impactor, or a hybrid design incorporating both electro-mechanical and pneumatic elements. A simplified diagram of such a design appears in FIG. 19. In the figure, an electrical motor (1901) is coupled to a slider (1903) via a linear motion converter (1906). The drive of the electrical motor is modulated, responsive to the control method described above by controlling motor current or voltage to modulate the drive force imparted to the slider (1903). The motion of the slider is further controlled according to the control method by modifying the upper (1902) and lower (1904) pneumatic chambers where control of air pressure is used to control the forward and reverse forces applied the slider. The slider is coupled to a tool head (1905), which may be a broach, and hence impacts the surgical site.

In an embodiment, the electro-mechanical impactor automatically modifies (e.g., reduces, or increases) the force of the delivered impact when approaching a pre-determined BSM state, where this state may additionally be set previously by the surgeon during planning of the procedure or during the procedure itself.

In yet another embodiment, the electro-mechanical impactor indicates the current BSM to the surgeon by automatically modifying the behavior of a control surface or haptic interface mounted upon or within the impactor. In another embodiment, the responsiveness of the trigger used by the surgeon to activate or de-activate the electro-mechanical impactor is modified in response to the direct or derived representation of the BSM communicated via interface (106). Here, responsiveness means the amount of force required to trigger the impactor or an induced vibration of the trigger directing the surgeon to adjust his or her impacting action.

In another embodiment, a control interface of the electro-mechanical impactor is created upon the handle or grip area of the impactor, responsive to the BSM or derived metric and located such that the surgeon can perceive haptically the BSM or derived metric and act accordingly.

The metric—such as a BSM, MTI, TI etc.—may also be stored during the procedure for subsequent analysis after the procedure to estimate one or more derived quality measures associated with the procedure. Alternatively, the sensory data (101-103) may be stored for subsequent non-real-time processing to generate the same quality measures. Such derived quality measures may include, for example, a measure of the probability of post-operative complications such as femoral fracture or looseness of fit of the prosthesis.

Note that in an embodiment, the apparatus described in FIG. 1 may be a stand-alone apparatus wholly deployed within or near to the operating room in which the procedure is occurring. Or it may be a distributed apparatus with, for example, a portion of the apparatus—such as sensors (101-103)—deployed within the operating room while other portions of the apparatus—such as processor (100)—are deployed remotely within the hospital hosting the operating room, or within a remote cloud computing center. In another embodiment, the apparatus may be wholly or partially integrated within the electro-mechanical impactor.

As illustrated in FIG. 2, generation of the broaching state metric (BSM) estimate is accomplished by the processor (100) through the method of applying of one or more mathematical, signal processing and/or machine learning algorithms or processes. It will be appreciated that not all the algorithms and processing stages described in FIG. 2 may be necessary, and that only a subset of the stages may be needed.

Note that while the invention is described preeminently in terms of a method and apparatus to determine the broaching state metric (BSM) of an orthopedic surgical site, broader application of the invention is readily envisaged. For example, the invention may be applied to a physical system observed by sensors, where the state of the physical system is inferable from those observations. As a more detailed example, the invention may be used to determine the state of a mechanical stamping machine used to form parts from raw material. In this example, by monitoring the impacts of the machine, the calibration of the machine, degree of machine wear, and other useful metrics can be determined.

### Time Interval Pre-Processor (TIPP)

In one embodiment, as shown in FIG. 2, the sensor data (101-103) is coupled, at a specified sequence of sampling instants according to a sensor sampling process, to the input of the time interval pre-processor (TIPP, 201). The sensor sampling process may be periodic or aperiodic in time and need not be identical for each sensor. In an embodiment, the sensor sampling interval is constant and uniform for all sensors.

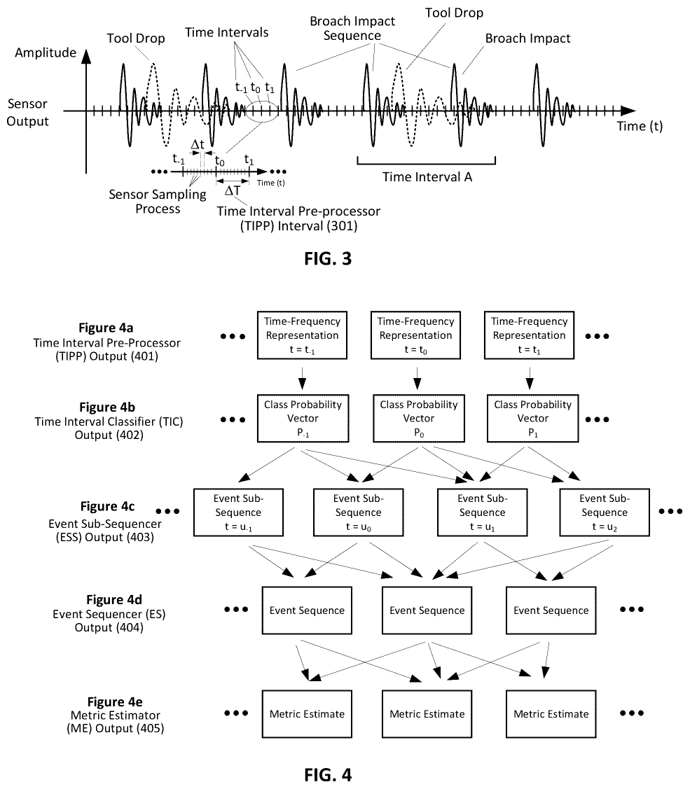

FIG. 3 shows acoustic data sampled at a uniform interval Δt. The output from a single acoustic sensor is shown, but data from more than one such sensor may be present. Notably, the acoustic data may comprise observations of several different types of acoustic events occurring within the operating room. For example, FIG. 3 shows a sequence of broach impacts that overlaps in time with two surgical tool drop events. Such events can occur, for example, when a surgical tool is replaced on a tray within the operating room, or when a tool is discarded into a waste receptacle after use. In the example of FIG. 3, the tool drop that occurs within Time Interval A would modify the total or sum time-domain envelope of the resulting composite waveform (i.e., the sum of the broaching and tool drop events) during each of the broach impacts that occur within Time Interval A, making detection of a broach impact acoustic event using envelope detection alone very difficult.

As shown in FIG. 3, the time interval pre-processor (TIPP, 201) transforms the data offered by the sensors (101-103) over time intervals of duration ΔT. In FIG. 3, the TIPP sampling interval ΔT (301) is shown to be uniform, but irregular sampling processes are also possible. Each TIPP interval ΔT comprises M sensor sampling intervals of duration Δt. TIPP sampling intervals occur at time tₖ=kΔT where k is an integer. Note that in what follows, for simplicity we refer generally to sensor data as acoustic data, but as previously discussed the data may also be strain or stress data and so on.

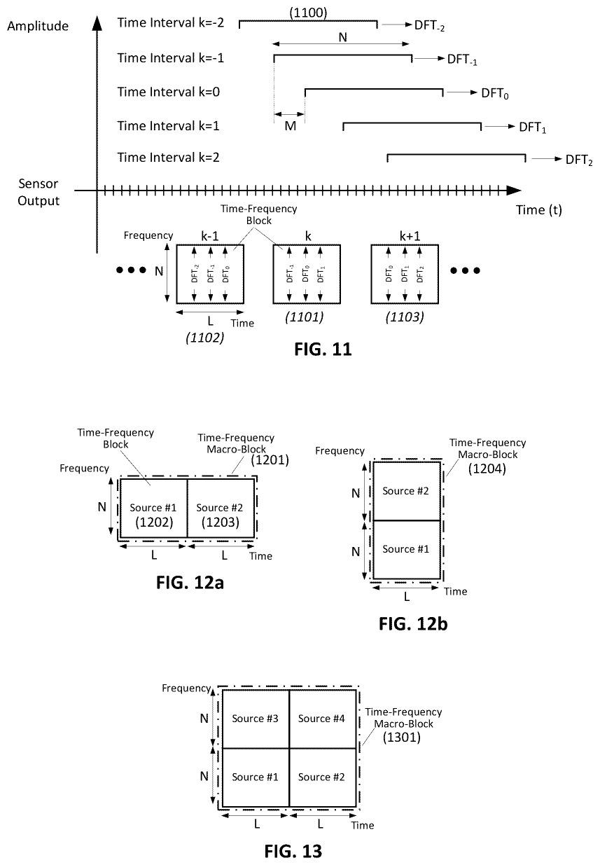

To preserve all the information contained within the audio record, and as shown in FIG. 11, the TIPP generates a sliding time-frequency representation of the sampled acoustic data. At each TIPP interval ΔT, the TIPP forms a length-N sequence (1100) of audio samples and executes a discrete Fourier transform (DFT), which may be based upon any well know DFT-windowing method such as a Hamming window etc., to generate a length-N frequency domain representation (other lengths are possible, but for convenience we focus on length-N). As shown in FIG. 11, that representation is then combined with the DFT of neighboring acoustic data sequences, each formed by shifting the acoustic data window (1100) by M samples, to form a time-frequency block representation (1101) corresponding to TIPP interval tₖ=kΔT. Typically, if the acoustic data is sampled at 48 kHz, the value of N may be set to 480 samples corresponding to 10 ms duration. The value of M may typically be set to 48 samples, corresponding to a 1 ms TIPP sampling interval ΔT. Other values of M and N are possible. The result is a sequence of time-frequency blocks (1101), (1102), (1103) each comprising L frequency-domain vectors of length N.

In an embodiment, the TIPP (201) further normalizes the signal representation in each time-frequency block. This aids signal scaling and intermediate value dynamic range management in subsequent processing stages. For example, if the DFT of the k-th length-N sequence (1100) is Hₖ (m) then the magnitude-squared value corresponding to each sample of DFTₖ is |Hₖ(m)|². In one embodiment, for the purpose of optimizing subsequent processing stages, |Hₖ(m)|² is normalized over each time-frequency block to have a specified mean and variance. Typical values are zero mean and unit variance. Each time-frequency block may also be further transformed into the logarithmic or log domain—that is, by modifying the value corresponding to each sample of DFTₖ to be 10 log₁₀|Hₖ(m)|². Further processing by the TIPP to achieve specific mean and variance in the log domain can then be executed. That is, the value of 10 log₁₀|Hₖ(m)|² over the block may be normalized to, say, zero mean and unit variance. In another embodiment, the TIPP may further transform the frequency axis of each time-frequency block through one or more linear or non-linear transformations. Well-known examples of such transformations include the Mel frequency mapping, Bark frequency mapping, and Mel Frequency Cepstrum mapping. Such transformations may alter the value of the frequency dimension N of each time-frequency block (1101-1103).

In a further embodiment, the TIPP (201) also equalizes the fundamental frequency response of the operating room. Operating rooms vary in dimension, installed equipment, wall coverings, etc. These factors can affect operating room acoustics. Microphone placement can also vary from operating room to room, or according to patient placement. Microphone vendors and designs may also vary, even within the set of sensors (101)-(103). This leads to a variation in the composite time-frequency response of the room, or more precisely, to the transfer function between the biomechanical system comprising the broach or prosthesis plus the femur and the acoustic sensor or sensors. It can be beneficial for the TIPP (201) to compensate for such variation so that subsequent processing stages observe more similar data that does not vary as much between rooms. As shown in FIG. 16, this can be done conveniently, for example, by applying an equalizing function, expressed as frequency response G(m), to each computed DFT to generate a modified or compensated DFT* whose frequency representation is H*ₖas follows:

H*ₖ(m)=G(m)Hₖ(m)

The resulting DFT* is then used in place of DFT in the time-frequency blocks and macroblocks. This equalizing operation can also be done in the time domain. The equalizing function G(m) can be computed using standard minimum mean-square error or zero-forcing equalizer design criteria. It is important however, to have a standard reference source signal against which to compute or train the equalizer response G(m). In an embodiment, this is done by deploying or making use of an existing known reference source within the operating room. Such a known reference signal may be any known signal, but broadband signals covering a wide frequency band are known to be beneficial in sounding the entire frequency structure of the acoustic channel from impact site to sensor. One convenient source of such a signal is the automated impactor device. Here, either an impact generated during the broaching process, or an impactor activation triggered when not in contact with the broach or prosthesis but while still in proximity to the broach or prosthesis and observable by the sensors (101-103) can be used. In an embodiment, this creates a standard acoustic reference against which the equalizing function G(m) can be computed.

In another embodiment, in cases where the TIPP is processing more than one acoustic sensor or source (101)-(103), the time-frequency block structure may be extended to support the additional source or sources by concatenating time-frequency blocks from different acoustic sources to form a time-frequency macro-block for subsequent processing. An example of this appears in FIG. 12A, which shows a time-frequency macro-block (1201) formed by concatenating two time-frequency blocks (1202, 1203) from distinctive sources along the time axis. Equivalently, as shown in FIG. 12B, the macro-block may be formed along the frequency axis (FIG. 12A, (1204)). A further example, where four acoustic sensors or sources are combined to generate a macro-block (1301), appears in FIG. 13. In what follows, for simplicity, we focus on the single sensor case and hence a single time-frequency block, but it will be understood that the same processing concepts apply equally to time-frequency macro-blocks comprising more than one time-frequency block.

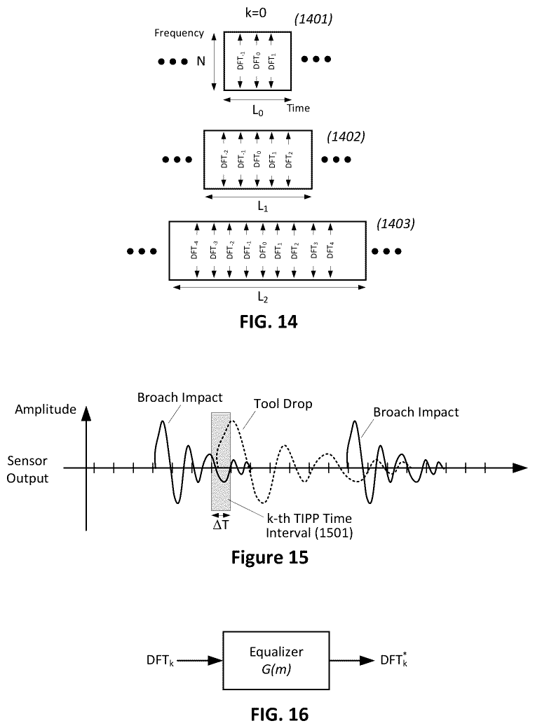

The TIPP may also construct time-frequency blocks of different dimension corresponding to the same time instant tₖ=kΔT for subsequent processing. An example of this embodiment appears in FIG. 14 which shows three (3) time-frequency blocks (1401), (1402), (1403) centered on t k although an arbitrary number of blocks may be so constructed. The time-frequency blocks have uniform dimension N in the frequency axis, but dimensions L₀, L₁ and L₂ respectively in the time axis. It is also possible to vary the frequency dimension N between blocks and to extend the same concept to form time-frequency macro-blocks of varying dimension in frequency, or in both time and frequency associated with the same sample interval tₖ.

Another embodiment is shown in FIG. 18. In this instance, the time-frequency blocks associated with time sample interval t k are stacked by the TIPP to form the macro-block (1801). In the example of FIG. 18, time-frequency blocks from three acoustic signal sources, (1801)-(1803) are stacked. It will be clear that the time-frequency block structures shown in FIGS. 11, 12A, 12B, 13, 14, and 18 can also be combined and hybridized in different combinations and variations.

### Time Interval Classifier (TIC)

Reverting again to FIG. 2, the output of the time interval pre-processor (TIPP, 201) in the form of time-frequency blocks (or macro-blocks) occurs at uniform time intervals tₖ=kΔT although, as stated previously, non-uniform TIPP sampling is also possible. The time-frequency blocks are next passed to the time-interval classifier (TIC, 202).

For each time instant tₖ, and corresponding k-th time interval of duration ΔT, amongst other functionality, the TIC estimates the probability that the k-th TIPP time interval forms part of an instance of one of a set of Q observable event types. Usually, but not always, an event corresponds to a physical event in the operating room leading to an acoustic or stress or strain or vibrational sensor response. As before, for simplicity, we focus on acoustic sensors and hence acoustic events, although as previously discussed, other sensor types are possible.

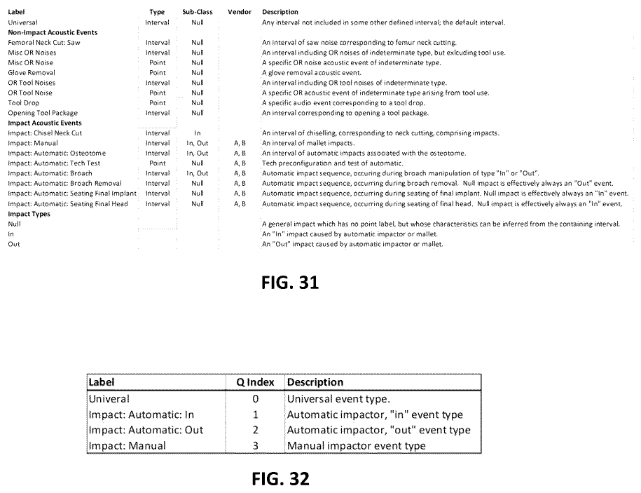

An example of this appears in FIG. 15, which shows a k-th TIPP time interval (1501) comprising a portion of two different acoustic events: a tool drop event and a broach impact event. An arbitrary number of event types may be defined to be included in the set of Q event types. The set Q includes an event type which denotes any event not otherwise identified or enumerated. This is referred to here as the universal event type and, for convenience, when enumerated is often referred to as the zeroth event type. FIG. 31 show an example of a set of event types.

Event types in FIG. 31 can be separated into two groups: non-impact related acoustic events and impact related acoustic events, although other groupings are possible. Impact related acoustic events are those events related to a broach impact. Events can also be separated into “interval” and “point” events. Interval events are events which have a prolonged duration and do not generally comprise a specific, individual event. An example of an interval event is an extended period of sawing e.g., for the purpose of cutting a femoral neck. Sawing is also an example of a non-impact related event. Point events are transient, discrete events, often of relatively short duration compared to interval events. An example of a point non-impact event is glove-removal, where an individual present in the operating room removes a glove. Another such example of a point non-impact event is a tool drop where, for example, a surgical instrument is replaced onto a table or discarded into a receptacle.

Acoustic events related to impacts appear as a second event group in FIG. 31. This event type group comprises point or interval events related to manual mallet or automated impactor impacts during chiseling or broaching activities. Some events are also subject to a directional sub-classification, such as ‘in’ or ‘out’ impact events, depending on whether, for example, a broach is being driven into or out of the femoral canal. The terms ‘in’ and ‘out’ can also refer to the configuration of an automated impactor when configured to drive in a forward or reverse direction. Further sub-classifications of events are possible—for example, by vendor type. In the example of FIG. 31, two impactor vendors A and B of the automated or manual impactors appear as event type sub-classifications.

In an embodiment, each member of the set of Q event types recognized by the time-interval classifier (TIC) are then defined and identified by combining one or more of the attributes of a scheme such as that of FIG. 31 in a sequential fashion, where the sequence may also represent a tree-leaf branching structure. For example, a member of the set of Q event types may be defined as a Tool Drop: Point event type, or an Impact: Manual: In: Vendor A event type, or an Impact: Automated: Broach: In: Vendor B event type, and so on, depending on which sub-classifications of FIG. 31 are determined to be useful or significant in a specific context. In an embodiment, these event type definitions can then be used as labels to be associated with each event type for the purpose of further processing. In an embodiment, these definitions or labels are used for the purpose of supervised training of a machine learning algorithm or procedure. Instances of each event type recorded in an operating room are tagged with these labels—either contemporaneously with the event or by subsequent analysis of a video recording of the operating room—and then used as the basis for supervised machine learning training. Any TIPP time interval portion of such audio data which is not tagged or labelled as one of the Q−1 event types other than the default universal event type, can be tagged or labelled as an event of universal type, and can be included in any subsequent training process with such a tag or label.

Amongst other tasks, in an embodiment, the Time Interval Classifier (TIC, 202) then estimates a length-Q class probability vector Pₖ whose i-th element Pₖ(i) denotes the probability that the k-th TIPP time interval comprises a portion of an instance of the i-th event type. As stated previously, it is important that all the time-frequency structure of each sensor observations i retained for processing, to ensure that all the information present is preserved. To maximize the accuracy and informativeness of the probability vector Pₖ it is important that the time interval classifier (TIC, 202) has access to a complete set of information concerning the current time interval rather than only e.g., a limited or pre-determined set of frequency bands or a feature derived from a such a set of bands, such as a local power estimate within a set of bands. Such approaches apply, and are limited by, classical concepts of human-perceptible filtering for specific frequency regions of each time-frequency block. They also tend to be restricted to linear processing operations. Instead, it is desirable and beneficial to be able to exploit any significant relationship between the elements of each of elements of each of the DFT vectors comprising the time-frequency blocks for the purpose of computing the event class probability vector Pₖ and consequently also for subsequently estimating the BSM.

Rather than being limited to linear operations, the universal approximation theorem shows that an appropriately constructed deep neural network (DNN), including non-linear processing or activation functions, can support arbitrary and complete mappings from the data Hₖ(m) comprising the time-frequency block to the class probability vector Pₖ. Such a neural network, operating across the entire time-frequency block processed by the time interval classifier (TIC, 202) can exploit relationships between all the time-frequency data Hₖ(m) rather than a limited sub-set of that data, such as particular frequency bands. This allows the TIC to identify non-obvious or hidden relationships in both the time and frequency domains between the N×L elements comprising the entire time-frequency block.

In one embodiment therefore, the time interval classifier (TIC, 202) is implemented as a deep neural network (DNN) comprising multiple partly or fully connected layers whose outputs are the weighted combination of the previous layer, subject to a non-linear transformation such as the rectilinear unit or sigmoid functions. Examples of deep neural networks may be described in I. Goodfellow et al., “Deep Learning”, MIT Press, ISBN: 9780262035613”, the entirety of which is incorporated herein by reference. A convolutional neural network (CNN) provides a means of achieving this. CNNs are rooted classically in the image processing problems of object identification and segmentation. In that context, the information in adjacent pixels is combined in a layer-by-layer fashion through operations such as local weighted combining, non-linear activation function processing, local averaging and pooling, spatial decimation, and down-sampling to construct image-spatial filters and to distill and compress the structure and information content of the source image. Examples of CNNs may be described in Y. Bengio, “Learning Deep Architectures for AI”, Foundations and Trends in Machine Learning. 2 (8): 1795-7. CiteSeerX 10.1.1.701.9550. doi:10.1561/2200000006. PMID 23946944, the entirety of which is incorporated herein by reference. In the current context, in some embodiments, the pixel intensity values may be replaced with the time-frequency block output of the TIPP (201), where pixel intensity values are replaced with |Hₖ(m)|² or 10 log₁₀|Hₖ(m)|² or any of the other TIPP outputs previously described. When used in the context of a CNN, it is the sometimes-hidden and not predefined relationships between the time-frequency block elements Hₖ(m) that are processed by the CNN, rather than image regions or segmented objects edges as in the case of image processing. High performance and computationally efficient examples of CNN designs that can be used for this purpose include DenseNet, VGG, Inception, amongst others. Examples of DenseNet may be described in G. Huang et al., “Densely Connected Convolutional Networks,” arXiv:1608.06993, the entirety of which is incorporated herein by reference. Examples of VGG may be described in K. Simonyan, “Very Deep Convolutional Networks for Large-Scale Image Recognition,” arXiv:1409.1556, the entirety of which is incorporated herein by reference. Examples of inception may be described in C. Szegedy et al., “Going Deeper with Convolutions,” arXiv:1409.4842, the entirety of which is incorporated herein by reference.

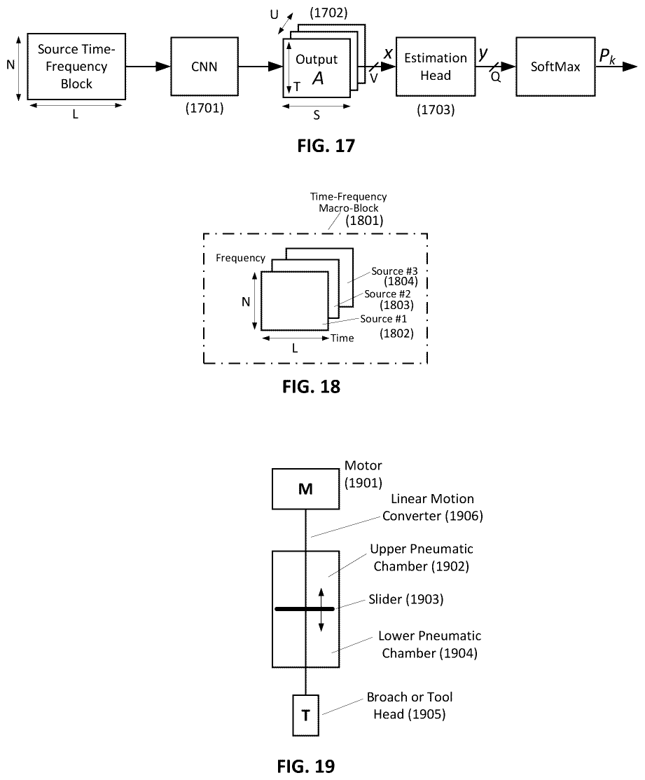

In one embodiment, as shown in FIG. 17, a CNN (1701) processes the time-frequency block. In general, the CNN outputs a hyper-dimensional data block (1702). For simplicity, we represent this in 3-dimensions, although higher dimensional representations are possible, with a resulting output block or tensor A (1702) of dimension [S, T, U], where the number of elements V of A is usually equal to V=S×T×U. The elements of A are then transformed for input into an estimation head (1703). This transformation can take form of a re-mapping, by row and column, of the multi-dimensional content of A to form a linear vector x of dimension V, although other re-mappings are possible. x is then processed by the estimation head (1703) comprising one or more neural network layers, to form Q-dimensional output y. In the case of a single layer estimation head, y=σ(Wx+b) where W is a matrix of dimension Q×V, b is a bias vector of length Q, and σ is a non-linear transformation such as the rectilinear unit or sigmoid function.

The output y of the estimation head (1703) is then processed by a SoftMax function, sometimes referred to as a normalized exponential function, to form vector Pₖ where the SoftMax operation has the form:

$${P}_{k} ( i ) = \frac{{e}^{y ( i )}}{{\sum }_{j = 0}^{Q - 1} {e}^{y ( j )}}$$

Here, Pₖ(i) and y(i) are respectively the i-th vector elements of Pₖ and y.

In one embodiment, training of the CNN structure (1701) of FIG. 17 is executed across the set of TIPP time intervals comprising a training set of intervals labelled with known acoustic event types. That is, each TIPP interval (ΔT, FIG. 3), is assigned a tag or label from the set of Q available tags or labels. This includes all portions of the audio record that do not correspond to specific instances of identifiable acoustic events. As previously described, for training, such intervals are assigned to the default universal event type.

Having so assigned each TIPP sample interval in a training audio record to one of the Q acoustic event types, the weights, bias terms, and any other adaptable parameter of the CNN (1701) and estimation head (1703) of FIG. 17 are optimized to minimize a loss function L. In one embodiment, the loss function is the cross-entropy (CE) loss function L_{CE}, sometimes referred to as the categorical cross entropy loss function, defined over the set of TIPP interval observations comprising the training set as follows:

$${L}_{C E} = {\sum }_{k} {\sum }_{i = 0}^{Q - 1} {T}_{k} ( i ) log [ {P}_{k} ( i ) ]$$

Here, Tₖ(i) is a binary variable, set to unit value if the k-th observation of the training

set of TIPP intervals is labelled with the i-th of the Q acoustic event type labels, else set to zero. In practice, other loss functions are also possible, including e.g., the weighted cross-entropy loss function which is sometimes used when significant imbalance exists in the number of members of each event type represented in the training set. The weights, bias terms and any other adaptable parameter of the CNN and estimation head of FIG. 17 are optimized according to the loss function L_{CE} by batching together subsets of the training set, estimating L_{CE} over that subset, and applying gradient estimation and backpropagation methods to iteratively estimate the adaptable parameters of the CNN.

In the case of multiple audio sources, in one embodiment, the dimensions of the CNN input in FIG. 17 are adjusted to match the dimensions of the time-frequency block or macro-blocks described previously. For example, the N×L block dimensions shown as input in FIG. 17 can be modified to have dimension N×2 L in the case of FIG. 12A, 2N×L in the case of FIG. 12B and 2N×2 L in the case of FIG. 13. Stacked time-frequency macro-blocks—such as the example of FIG. 18—can be handled by making use of image processing CNN architectures which process individually the separated color planes used in color images. That is, such image processing CNNs are designed to accept 3-plane red-green-blue (RGB) or chroma (YCbCr) N×L images. In an embodiment, these CNN designs are used to accept into each input color plane as input an N×L time-frequency block drawn—as shown in FIG. 18—from a stack of such blocks. A non-uniformly dimensioned stack of blocks such as that shown in FIG. 14 can also be processed in this way.

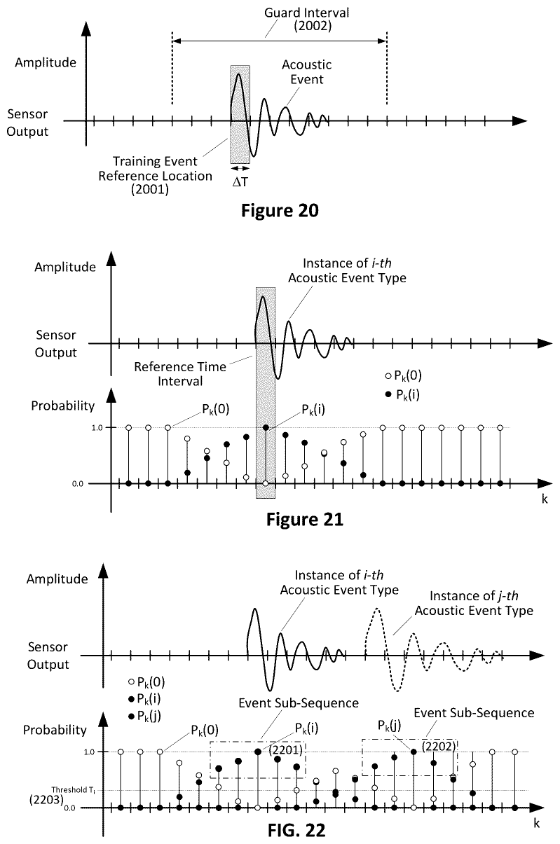

When constructing the training set of labelled TIPP time intervals, in one embodiment the labelled acoustic event is assigned to a reference TIPP time interval (2001), as show in FIG. 20, with the time-frequency block associated with that interval defined as the time-frequency block associated with that acoustic event and label. In order to preserve the training view of the event, as also shown in FIG. 20, a guard interval (2002) is assigned in proximity to the reference TIPP interval (2001). Intervals selected from the audio record for the purpose of training for classification of the universal acoustic event are then excluded from being drawn from this guard region around any instantiated training acoustic event. This avoids training cross-contamination between event types.

As previously discussed, after machine learning training is complete, machine learning inference is executed by the TIC (202) using the trained processing system of FIG. 17. Processing is executed in turn on the time-frequency block associated with each TIPP time interval, resulting in a length-Q vector Pₖ corresponding to each kth TIPP time interval. FIG. 21 illustrates conceptually the behavior of Pₖ for the case when there is a single acoustic event type present (labelled in FIG. 21 as an example of the ith acoustic event type) along with the universal event labelled as the zeroth acoustic event type. The probabilities Pₖ(0) and Pₖ(i) corresponding to the zeroth and ith acoustic event respectively for each time interval are also shown in FIG. 21. As the source time-frequency block approaches in time to the location of the reference time interval for the instance of the ith acoustic event type present, there is an increase in the probability Pₖ(i) assigned to the time interval that that associated acoustic event is present. There is a corresponding decrease in the probability Pₖ(0) that the time interval is associated with the universal acoustic event.

### Event Sub-Sequencer (ESS)

Reverting again to FIG. 2, in one embodiment, the output of the time interval classifier (TIC, 202) is input to the event sub-sequencer (ESS, 203). The primary purpose of the event sub-sequencer is to associate or group nearby or proximal and not necessarily adjacent TIPP intervals into groups termed event sub-sequences that are attributed to or linked with a specific acoustic event. This recognizes that the duration of acoustic events, whether point or interval, typically span more than one TIPP time interval. For example, if the TIPP time interval is 1 ms, and a typical point acoustic event such as an impact has a duration when measured at the output of the TIC of 40 ms, the impact acoustic event will span 40 TIPP time intervals.

FIG. 22 illustrates how, in one embodiment, the event sub-sequencer (ESS, 203) associates time intervals with an acoustic event. In this simplified example, two instances of acoustic events are shown, drawn respectively from the ith and jth event types. As previously discussed, the universal acoustic event—denoted as the zeroth event type—is also present.

The lower graph of FIG. 22 shows the TIC output probability vector Pₖ, decomposed into respective Q=3 audio types, that is, into probabilities Pₖ(0), Pₖ(i), and Pₖ(j). In one embodiment, in FIG. 22 the ESS identifies the event sub-sequence associated with the event of ith type by associating with the corresponding event sub-sequence (2201) those time intervals where Pₖ(i) exceeds Pₖ(0). That is, where the probability of the ith event exceeds that of the universal event. Similarly, as shown in FIG. 22, the event sub-sequence associated with the event of jth type is constructed by associating or grouping (2202) those time intervals where Pₖ(j) exceeds Pₖ(0). In another embodiment, the probability of an ith event must exceed that of all other event types in order to be designated as a portion of an ith event type.

Additional rules may be applied, in different combinations, to govern how TIPP time intervals are associated with an event sub-sequence. In an embodiment, as shown in FIG. 22, when constructing an event sub-cluster of the ith type, the probability Pₖ(i) of associated time intervals can also be required to exceed a threshold Tᵢ(2203) (the same or a different threshold Tⱼ may apply to the jth event type and so on). Another embodiment of a rule applicable to event sub-sequence construction is to specify a minimum number N_{ess,min}(i) of TIPP time intervals that must be candidates to be associated with a candidate ith type event sub-sequence to declare it a valid ith type event sub-sequence.

Noise or other artifacts can influence Pₖ. Accordingly, as an optional first stage of processing, in an embodiment the event sub-sequencer (ESS) filters the probability vectors Pₖ in the time-domain prior to event sub-sequence construction. For this purpose, a low-pass filter is used, which can conform to any number of well-known filter architectures, including finite impulse response (FIR) and infinite impulse response (IIR) designs. In one embodiment, a symmetric, odd-length, FIR low-pass filter is used, which provides the benefits of a linear-phase response, or equivalently, a simple and well-defined delay through the filter. That is, if the filter length in taps is M, the filter delay will be equal to (M−1)/2.

Defining the FIR filter taps as v (k), k={0, M−1} each of the Q elements of the probability vector Pₖ is processed to generate a filtered, smoothed output vector N comprising the elements P*ₖ(i) computed according to:

$${P}_{k}^{*} ( i ) = {\sum }_{m = 0}^{M - 1} v ( m ) {P}_{k - m} ( i )$$

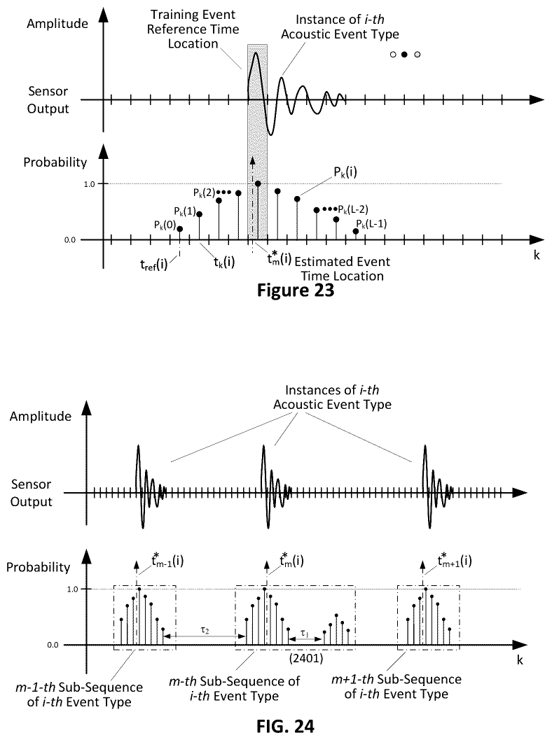

In one embodiment, when the probability vector Pₖ (or its filtered version P*ₖ may be used in all operations that follow involving Pₖ) indicates the presence of an acoustic event, and the event sub-sequencer (ESS, 203) has constructed an associated event sub-sequence, the event sub-sequencer (ESS, 203) also generates an estimated event time location for the event. This is done by time-weighting the probability vectors Pₖ(i) associated with an event of ith type. As shown in FIG. 23, the estimate t*ₘ(i) of the time location of the mth detected acoustic event of the ith type can be computed as:

$${t}_{m}^{*} ( i ) = {t}_{ref , m} ( i ) + {\sum }_{k = 0}^{L - 1} {P}_{k} ( {t}_{k} - {t}_{ref , m} ( i ) )$$

Here t_{ref,m}(i) is the reference time location associated with the onset of the mth detected acoustic event of the ith type. For convenience in processing, t_{ref,m}(i) may be quantized in time to the nearest TIPP time interval.

FIG. 24 further illustrates the generation of multiple such event sub-sequences and associated reference time locations, enumerated in the example of FIG. 24 as event sub-sequence locations t*ₘ₋₁(i), t*ₘ(i), and t*ₘ₊₁(i) respectively corresponding to the m−1th, mth, and m+1th instances of event sub-sequences of the ith type. Although not shown in FIG. 24, corresponding sets of event subsequences are generated for any event type j. The exception is the universal event type. That remains the default event type associated with any TIPP interval not otherwise associated with a non-universal event type.

FIG. 24 also illustrates a further class of rules which, according to one embodiment, are included in the process of event sub-sequence construction. In this example, during construction of the mth event sub-sequence of FIG. 24, two event sub-clusters which are close in time are merged into a single event sub-sequence. More generally, as illustrated in FIG. 24, if two event sub-sequences lie no further than τ₁ apart in time, then they are merged into a single sub-sequence (2401). If, as also illustrated in FIG. 24, two event sub-sequences are separated by a distance equal to or greater than time τ₂ which is greater than or equal to τ₁ then the corresponding event sub-sequences are not merged.

In another embodiment, in addition to the estimated event time location associated with an event sub-sequence, the event sub-sequencer may also associate a confidence, composite probability, soft metric or soft decision with each event sub-sequence. Taking the example of FIG. 23 again, the soft metric Sₘ(i) associated with the mth event sub-sequence of ith type may be computed as:

$${S}_{m} ( i ) = {\sum }_{k = 0}^{L - 1} {w}_{i , k} {P}_{k}$$

Here, w_{i,k} represents a set of weights associated with soft metric computation of the ith acoustic event type. In one embodiment, all the weights w_{i,k} may be set to unit value, meaning that the soft metric associated with the event sub-sequence is simply the sum of the TIPP interval probabilities associated with that event sub-sequence. Another embodiment forms the mean of the interval probabilities, that is, sets w_{i,k}=1/L, and so on. This embodiment permits a further exemplary rule concerning the generation of event sub-sequences which is to discard any sub-sequence whose soft metric is less than some, potentially ith event type dependent, threshold S_{T,i}. That is, the ESS discards event sub-sequences where:

Sₘ(i)≤S_{T,i}

In the interests of reducing computational complexity, the ESS need not construct sub-sequences for acoustic event class types that, even if defined, are not of interest or required in subsequent processing stages.

### Event Sequencer (ES)

Referring again to FIG. 2, in one embodiment, the output of the event sub-sequencer (ESS, 203) is input to the event sequencer (ES, 204). In an embodiment the primary function of the event sequencer is to associate one or more acoustic event sub-sequences with an acoustic event sequence.

Of special interest for the present purpose is the case where the acoustic event sequence is a sequence of broach impacts. Here, as previously described, a broach impact corresponds to an individual blow by a mallet or impactor onto the femoral broach. A broach impact sequence, or more simply an impact sequence, is a sequence of such impact events. Note that when executing a THA, the surgeon will typically execute the broaching function in a sequence of broaching steps. The surgeon may execute one or more sequences of broach impacts, then change or re-position the broach, and then execute one or more further sequences of broach impacts. Accordingly, from the perspective of the broaching process, a surgical procedure may be constructed from a series of broach impact event sequences.

More generally, acoustic event sequences comprise a set of one or more acoustic event sub-sequences of the same or different acoustic event type. The event sequencer (ES, 204) therefore constructs event sequences of interest from component event sub-sequences generated by the event sub-sequencer (ESS, 203). In one embodiment, a first stage of selection of acoustic event sub-sequences as candidates is to assemble into an acoustic event sequence those event sub-sequences admissible to a target event sequence. The set E of such admissible events may be, for example, those event sub-sequences comprising a common ith event type. Other possible sets E may be defined. For example, in one embodiment acoustic event sub-sequences of type Impact: Automated: Broach: In and Impact: Automated: Broach: Out are admissible into a single event sequence since both even types can form part of a single impact sequence. Similarly, an event sequence of interest might be restricted to a single acoustic event type such as only the event type Impact: Automated: Broach: In when only “in” impact events are of interest.

Accordingly, in an embodiment, a first means of determining those event sub-sequences that should comprise an event sequence is to consider only those event sub-sequences comprising the set E. There may multiple such sequence definitions of interest, with set Eₚ defining the set of event sub-sequences admissible to the pth type of event sequence.

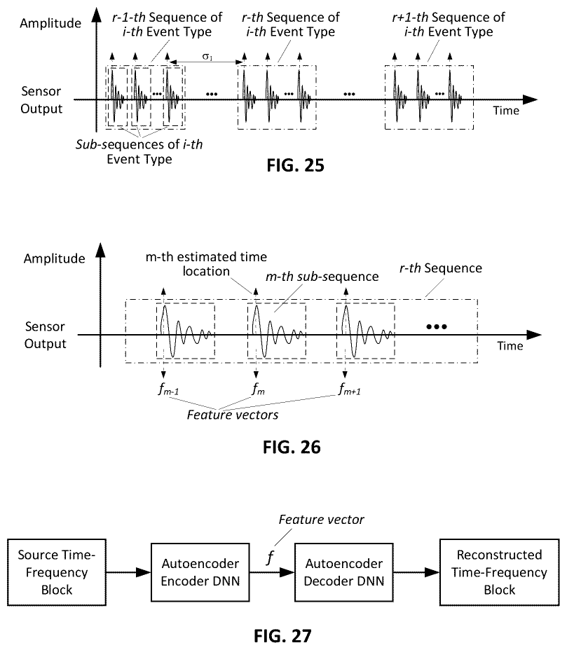

In another embodiment, event sub-sequence construction is performed by combining those event sub-sequences of interest (e.g., which are part of the set E) which are also separated in time by less than some maximum distance or delay. This is illustrated in FIG. 25, where event sub-sequences of a type included in the set E are combined into a single sequence where combined event sub-sequences have a time separation between estimated sub-sequence event time locations of less than or equal to σ₁. In one embodiment, event sub-sequences with an associated soft metric less than a threshold S_{T,i} are discarded from such combination.

The event sequencer may also exploit meta-data concerning the nature of the set E, and specifically, the relationships between members of the set E. In an embodiment, taking again the example set comprising the acoustic events Impact: Automated: Broach: In and Impact: Automated: Broach: Out, the event sequencer exploits the knowledge that an event sub-sequence cannot simultaneously correspond to both an “in” and an “out” event since the design of the automated impactor means it can be in only one of those states at a time. The event sequencer then selects the event sub-sequence with largest soft metric.

### Metric Estimator (ME)

Referring again to FIG. 2, in an embodiment, the output of the event sequencer (ES, 204) is coupled to the input of the metric estimator (ME, 205). The metric estimator (205) accepts feature vectors or soft information generated by any or all the preceding stages and processes that input to generate a metric indication. When used for the purpose of special interest here, the metric estimator (ME, 205) may generate a BSM indication.

In an embodiment, a metric estimate is generated at any required time across the length of an associated event sequence. In an embodiment, it is appropriate to generate a metric estimate corresponding to each event sub-sequence comprising the event sequence. In the specific case of the THA implant broaching process, this means generating a metric estimate, or more specifically a BSM, at each impact event comprising a sequence of such events.

In an embodiment, as shown in FIG. 26, this is done by first generating within each event sequence a feature or embedding vector fₘ derived from the time-frequency block centered at the mth estimated event sub-sequence location. In machine learning, a feature vector is a D-dimensional vector which describes an associated image, body of text, object, or other source of information. Note that it is possible to generate more than one feature vector corresponding to the mth estimated event sub-sequence location by computing other reference times lying within the time-interval spanning an event sub-sequence, sourcing the associated time-frequency blocks, and then generating the further associated feature vectors. For simplicity, we focus here on the case of a single feature vector per event sub-sequence.

Feature vectors can be generated or encoded or embedded in several ways. In an embodiment, an autoencoder is used. FIG. 27 shows the structure of a basic autoencoder used in the present application. In FIG. 27, time-frequency blocks are offered to the autoencoder encoder-decoder deep neural network (DNN) pair. In an embodiment, the weights, biases, and other adjustable parameters of both the encoder and decoder components of the autoencoder are then trained to minimize the mean-square error (MSE) between the original time-frequency block and the reconstructed time-frequency block generated by the decoder portion of the autoencoder. Other autoencoder training criteria can also be used. The output of the encoder portion of the autoencoder forms the feature vector f. It is from this feature vector that the decoder portion of the autoencoder subsequently derives its reconstruction of the source time-frequency block. Accordingly, the feature vector f can be said to contain a representation of the information present in the original time-frequency block. With appropriate design of the autoencoder, including using the MSE criterion, in the embedding space spanned by the feature vector, the distance d (fₘ, fₙ) between the feature vectors encoded from two distinctively different time-frequency blocks (blocks derived, say, from impactor or mallet strikes respectively) is proportional to the degrees of acoustic-informatic difference between the two blocks.

The architecture of the autoencoder encoder and decoder typically comprise deep neural networks (DNN), including convolutional neural network (CNN) structures, although there are important evolutions of this core architecture, including variational autoencoders. Importantly for the present purpose, and as shown in FIG. 26, in an embodiment the autoencoder encoder is run to generate a feature vector fₘ at each mth estimated time location corresponding to an event sub-sequence within an event sequence.

Typically, the separation between event sub-sequences within an event sequence is 250 ms, which is 250× greater than the typical TIPP time interval of 1 ms. Accordingly, the rate at which the autoencoder encoder DNN is executed to encode feature vectors fₘ is much less than the rate at which the time-interval classifier (TIC, 202) executes the CNN of FIG. 17 for time-frequency classification purposes. Note that, after training, the decoder portion of the autoencoder is not required in the current application.

This presents two alternative embodiments for the autoencoder design of FIG. 27. In a first embodiment, the autoencoder encoder DNN of FIG. 27 is distinct from, and may be much more computationally complex than, the CNN of FIG. 17 used for classification purposes. In one embodiment, the autoencoder DNN is a more computationally complex CNN than the CNN of FIG. 17, and may include for example, many more neural network layers than the CNN of FIG. 17. The MSE criterion previously defined can be used to train this autoencoder and therefore to generate fₘ.

In a second embodiment, provided the event sub-sequence time location is, as previously described, quantized to the nearest TIPP time interval, the TIC classifier structure of FIG. 17 may be re-used to generate fₘ. That is, the vector x in FIG. 17 that is processed by the estimation head to generate the acoustic event class probability Pₖ for the TIPP interval corresponding to the event sub-sequence can be re-used as fₘ. In this case, the embedding space spanned by fₘ is one in which the vector x of FIG. 17 has been trained to support TIC classification, and so necessarily encodes information about the kth time-frequency block. This approach has lower complexity since vector x is re-used, although this may reduce the informativeness of the feature vector.

Importantly, in both embodiments for the generation of the feature vector fₘ, pre-selection of specific time intervals or frequency bands within the time-frequency block is not performed. Rather, all the information that can be encoded into the feature vector is beneficially retained from the time-frequency block, with the important regions of the time-frequency block left to the machine learning process to determine.

Also, as stated previously, in forming the BSM, it is important to make use not only of the information contained within the time-frequency blocks comprising an event sub-sequence, and therefore embedded in the corresponding feature vector, but also to incorporate all the information contained within the set of event sub-sequences comprising the event sequence. In other words, the BSM should be estimated by considering all the feature vectors fₘ comprising the event sequence. Further, within an event sequence, it is important to recognize that the BSM may evolve and change between the start and the end of the event sequence, as the physical state of the surgical site changes. This may occur, for example, as the broach or prosthesis penetrates further into the femoral canal.

In an embodiment, this can be achieved by designing the metric estimator (ME, 205) using stateful machine learning algorithms that have an awareness of state or which can capture sequential behavior. Examples of such algorithms include recurrent neural networks (RNN), such as the long-short term memory algorithm (LSTM), the gated recurrent unit algorithm (GRU) or transformer architecture (TA). Examples of LSTM may be described in S. Hochreiter, J. Schmidhuber, “LSTM can Solve Hard Long Time Lag Problems”, Advances in Neural Information Processing Systems 9. Advances in Neural Information Processing Systems, the entirety of which is incorporated herein by reference. Examples of GRU may be described in K. Cho at al., “On the Properties of Neural Machine Translation: Encoder-Decoder Approaches”, arXiv:1409.1259, the entirety of which is incorporated herein by reference. Examples of TA may be described in A. Vaswani et. Al, “Attention Is All You Need”, arXiv:1706.03762, 12 Jun. 2017, the entirety of which is incorporated herein by reference. In what follows, we focus on the GRU, but other such algorithms can be equally applied. Note also that variants of GRU architecture exist, mainly designed to achieve lower computational complexity. For simplicity we focus on a single example.

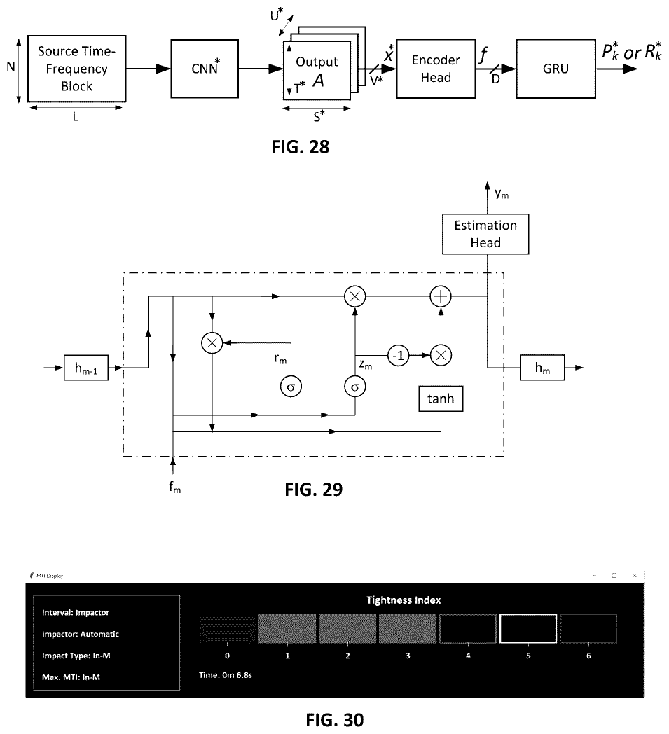

FIG. 28 shows an embodiment of GRU architecture. As shown in FIG. 28, a CNN* processes the time-frequency block. In general, the CNN* outputs a hyper-dimensional data block. For simplicity, we represent this in 3-dimensions, although higher dimensional representations are possible, with a resulting output block or tensor A of dimension [S*, T*, U*], where the number of elements V* of A is usually equal to V*=S*×T*×U*. The elements of A are then transformed for input into an encoder head. The encoder head, which may be, for example, a single layer neural network, or an autoencoder, or the encoder portion of an autoencoder, among other methods, modifies the dimension of the tensor output A. This is most usually a reduction in dimension, but other changes in dimension are possible. The dimensionally-modified output tensor f is then input into the GRU.

The internal GRU architecture appears in FIG. 29. In an embodiment, the GRU hidden state hₘ represents the state of the physical structure being assessed by the BSM. At each iteration of the GRU, corresponding to each event sub-sequence within an event sequence, the GRU hidden state hₘ₋₁ output by the prior iteration is updated by the new feature vector fₘ. In the process of updating according to FIG. 29, the GRU processes the feature vector via the non-linear activation function a to generate the reset gate rₘ update gate zₘ. In the case of the GRU these gates control the problem of vanishing or exploding gradients which can occur when dealing with long event sequences. The updated GRU state in FIG. 29 is represented by hₘ. As shown in FIG. 29, as well as being output towards the next iteration, the updated hidden state hₘ is provided as input to an estimation head which processes the updated hidden state to generate an estimate yₘ in of the BSM. In an embodiment, the estimation head has the same form as previously discussed. That is, yₘ=σ(Whₘ+b) where a is a general non-linear activation function (not necessarily the same as that used within the GRU), and where W and b are a trainable weight matrix and bias vector respectively.

Training of the GRU of FIG. 29 can be accomplished in several different ways. In a first embodiment, the loss function against which the trainable elements of the GRU are adapted can be a minimum square error (MSE) loss function. That is, if the BSM at the mth event sub-sequence is referred to be BSMₖ, the MSE loss can be computed on a batch basis as the expectation of:

L_{MSE}=[BSMₘ−yₘ]²

Similarly, BSMₖ may be defined to be selected from one of a finite set of BSM metrics or categories. In an embodiment the BSM is be defined to be drawn from the set BSMₖ∈B={0, 1, 2, 3, 4, 5} where, as previously discussed, the set B of categories ranges from 0 corresponding to a BSM indicating a loose fit to 5 corresponding to a BSM indicating a tight fit.

Initialization of the GRU hidden state—that is, the determination of h₀—can be done in several different ways. In one embodiment, h₀ can be set to zero or to a random vector. In another embodiment h₀ can be set to the final hidden state of a preceding event sequence, or alternatively a function of that final hidden state, if such a preceding event sequence exists.

Note also that the estimated metric value y in can correspond to the same event sub-sequence as the current feature vector fₘ but this is not necessary. For example, the GRU may be configured (including during training) to compute y_{m-N} after receipt of feature vector fₘ. That is, to include the influence of event sub-sequences, feature vectors and impacts observed before, during and after the time interval for which the BSM is to be estimated rather than just before and during. Provided the value of N is reasonably small—a value of, say, 3-4—the delay in delivering the BSM estimate to the surgeon is acceptable.

In an embodiment, during training of the GRU, based on his or her deep experience, the surgeon can create impacts corresponding to broach-bone system states of known or constant BSM value. The surgeon can then enable the announcement, annotation or labelling of the assessed BSM metrics while the physical state of the system comprising the broach and femur is held at or close to a constant state. In an embodiment, the surgeon uses his or her skill and experience to estimate the BSM value or category to assign to the current broach-bone system state rather than simply announcing, say, the distance a broach has progressed into the femoral canal. Here, the BSM metric that the surgeon announces may conform to a definition and scale known to the surgeon and/or adopted from a professional body or industry norm.

In an embodiment, such generated impacts or impact sequences corresponding to known or constant BSM, or iso-metric or iso-BSM, intervals are then associated with known or iso-metric state descriptors or labels for use in supervised GRU training, using for example the loss functions defined previously, and so train a machine learning system to identify BSMₖ during inferencing. Such a known or iso-metric state may also be identified and used for training purposes in an analysis executed after the procedure of sensor data recorded during the procedure.

Semi-supervised or unsupervised learning is also possible. In an embodiment, the mth GRU hidden state hₘ is used to predict a characteristic of a future event sub-sequence. Specifically, yₘ in FIG. 29 is trained to predict a characteristic of the dth future impact, where d typically has a value of 1—i.e., it is the characteristic of the next impact that is to be estimated. In an embodiment, the characteristic is determined by categorizing the next impact as drawn from the set B={‘in’, ‘out’, ‘none’}, where ‘in’ means the next impact is predicted to be an automated impactor ‘in’ impact, the ‘out’ designation means the next impact is predicted to be an ‘out’ impactor impact, and ‘none’ corresponds to a prediction that there will be no impact, or in other words, impacting will terminate.

In this way the end of the impactor sequence can be trained and then predicted and can be communicated to the surgeon via the human interface (104) as a recommendation to terminate impacting. Training for such categories requires no annotation or labeling by the surgeon, since it is clear from observation when each member of the set B occurs. This is therefore a form of unsupervised or semi-supervised learning.

### Processing Examples

FIG. 4, including FIGS. 4a-4e, shows a further representation of the resulting processing sequence of FIG. 2 for a particular embodiment when a single 1D acoustic, stress or strain sensor is present. FIG. 4a shows the output (401) of the pre-processor comprising the time-frequency representation centered on each TIPP time interval. FIG. 4b shows the TIC output (402) for each TIPP interval of the probability vector P of length Q. FIG. 4c shows the event sub-sequence outputs (403), while FIG. 4d shows the event sequencer outputs (404), and FIG. 4e shows the metric estimator outputs (405).

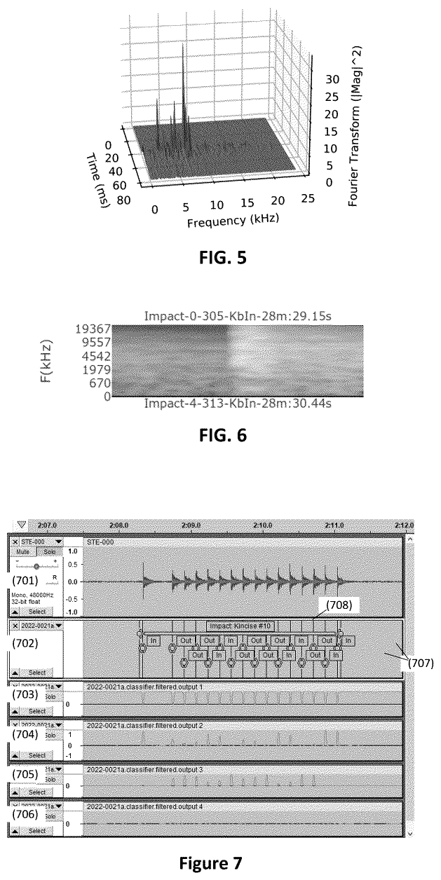

FIGS. 5-10 illustrate one embodiment of the invention in more detail based on recorded real-world data.

FIG. 7 shows the output (701) of an audio microphone placed in proximity to the surgical site. The output (701) is processed by the TIPP to generate time-frequency views such as those shown in FIGS. 5 and 6. FIG. 5 shows an isometric view of the time-frequency block with a linear frequency scale, while FIG. 6 shows a block as an intensity image, with intensity representing power expressed in decibels using a nonlinear scale frequency scale such as that defined by the Mel scale.

The time interval pre-processor output is provided at 1 millisecond (1 ms) intervals to the time interval classifier (202). FIG. 7 shows the output of the TIC (202) and specifically shows the estimated probability that each 1 ms interval corresponds to one of four impact event classes of FIG. 32, of respective types a) the universal event (703), b) an automated impactor ‘in’ impact event (704), c) an automated impactor ‘out’ impact event (705), or d) a manual or mallet impact event (706).

These class probabilities are collected into a single vector corresponding to each 1 ms interval. Note that an ‘in’ impact event occurs when the automated tool is configured to advance the broach further into the femoral canal, while an ‘out’ event occurs when the automated tool is configured to withdraw the tool from the femoral canal.

The class probability vectors are then processed by the event sub-sequencer (203) and event sequencer (204) to generate the sequencer output (404, 702) where the final designation of each impact to belong to one of the four event classes is executed. The estimated acoustic class designations—such as ‘in’ or ‘out’—may appear as labels (707) assigned to each impact event. The event sequencer (204) further automatically identifies each sequence of impacts as belonging to a specific sequence including an enumerated value identifying the sequence number within a larger set of sequences.

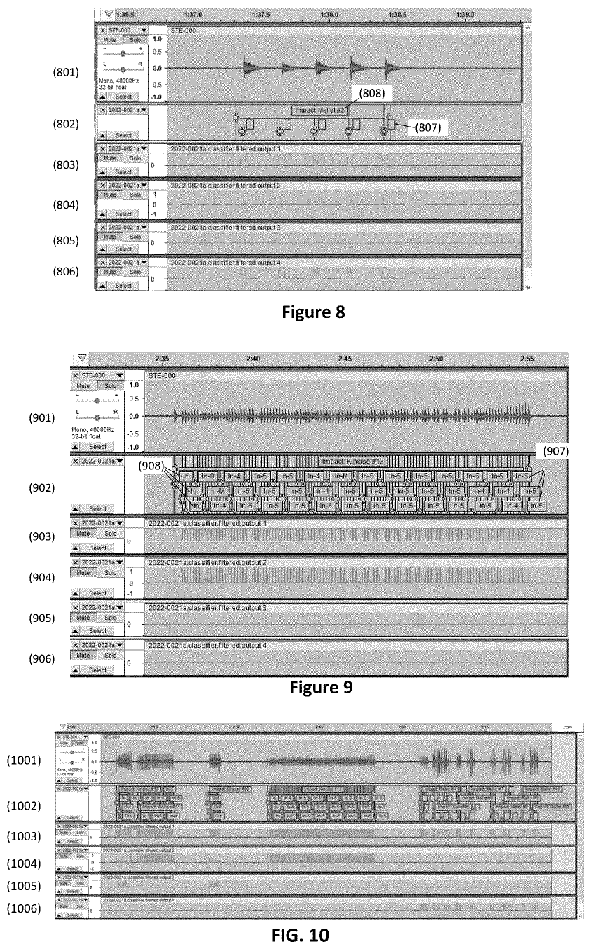

FIG. 8 shows a further example of event sequence identification (808), this time corresponding to a sequence of manual or mallet impacts. In this example, the mallet is nominally not specifically internally configurable to deliver ‘in’ or ‘out’ impacts and so each mallet impact event sub-sequence is not further labelled (807) as ‘in’ or ‘out’.

FIG. 9 shows the effect of the further processing by the metric estimator (205) to generate a metric associated with the BSM. In this example, each ‘in’ impact is further qualified as belonging to one of four values of BSM arranged along the scale of 0 (zero) to 5 (five) where 0 reflects the least tight value in a BSM or Mast tightness index (MTI) scale and 5 represents the tightest value. The BSM or MTI value assigned is a member of the set {0, M, 4, 5}, where ‘M’ corresponds to a mid-MTI value including values 1, 2, or 3. Other class sets can be configured. Alternatively, the classifier can be configured as a regressor and estimate the BSM or MTI as a value in the range 0-5.

Note that the BSM of the first few ‘in’ impacts in the sequence (908) is not generated since the GRU estimator used to generate the BSM or MTI value has not yet reached a stable estimate.

FIG. 10 shows multiple sequences corresponding to either automated or manual impacts with corresponding sequence and BSM/MTI labelling.

A number of variations are possible on the examples and embodiments described above. Accordingly, the logical operations making up the embodiments of the technology described herein are referred to variously as operations, steps, objects, elements, components, layers, modules, or otherwise. Furthermore, it should be understood that these may occur in any order, unless explicitly claimed otherwise or a specific order is inherently necessitated by the claim language.

Although several example embodiments have been described in detail above, the embodiments described are examples only and are not limiting, and those skilled in the art will readily appreciate that many other modifications, changes, and/or substitutions are possible in the example embodiments without materially departing from the novel teachings and advantages of the present disclosure. Accordingly, all such modifications, changes, and/or substitutions are intended to be included within the scope of this disclosure as defined in the following claims.

1. A method for determining a state of a bone or skeletal structure, comprising:
classifying a characteristic sensory signal observed during a surgical procedure, wherein the characteristic sensory signal comprises at least one of a stress, strain, haptic, tactile, or acoustic sensory signal;
isolating at least one sub-sequence or sequence of the classification of the characteristic sensory signal;
generating a metric associated with the state of the bone or skeletal structure based on the at least one sub-sequence or sequence.

2. The method of claim 1, wherein the state of claim 1 is a state of a reaming or broaching process.

3. The method of claim 1, wherein the state of claim 1 is a tightness associated with a reaming or broaching process.

4. The method of claim 1, wherein the metric is a broaching state metric (BSM), tightness index (TI), or Mast Tightness Index (MTI).

5. The method of claim 1, wherein the surgical procedure is a total hip arthroplasty procedure.

6. The method of claim 1, further comprising indicating the metric via a human interface.

7. The method of claim 1, wherein the human interface comprises a visual display comprising a meter, an audio interface or a tactile or haptic interface.

8. The method of claim 1, wherein the characteristic sensory signal results from impacts delivered by a manual or electro-mechanical impactor device.

9. The method of claim 1, further comprising determining a quality measure associated with the procedure based on the metric.

10. The method of claim 1, wherein generating the metric comprises generating the metric using a recurrent neural network (RNN), wherein the RNN comprises a long short term memory (LSTM) algorithm, a gated recurrent unit (GRU) algorithm, or a transformer algorithm.

11. A method of training a system to determine a state of bone or skeletal structure, comprising:
classifying a characteristic sensory signal observed during a surgical procedure, wherein the characteristic sensory signal comprises at least one of a stress, strain, haptic, tactile, or acoustic sensory signal;
decomposing the surgical procedure into one or more iso-metric states such that the characteristic sensory signal is acquired and associated with a descriptor or label of the one or more iso-metric states.

12. The method of claim 11, wherein the one or more iso-metric states correspond to the state of bone or skeletal structure during the surgical procedure.

13. The method of claim 11, wherein the descriptor or label and corresponding characteristic sensory signal are used to train a machine learning system.

14. An apparatus for determining the state of bone or skeletal structure based on the classification of characteristic stress, strain, haptic, tactile, or acoustic sensory signals observed during a surgical procedure, comprising:
one or more sensors; and
one or more processors, comprising at least one of random access memory (RAM) or read only memory (ROM).

15. The apparatus of claim 14, further comprising one or more human interfaces.

16. The apparatus of claim 14, where the apparatus is configured to operate during the surgical procedure.

17. The apparatus of claim 14, where the apparatus is configured to determine the state of the bone or skeletal structure after the surgical procedure.
